(19) United States
(12) Patent Application Publication
(10) Pub. No.: US 2006/0160086 A1
(43) Pub. Date: Jul. 20, 2006

(54) Method for continuous detection of an analyte, trifunctional detecting reagent used and detecting device

(75) Inventors: Herve Volland, Orsay (FR); Christophe Creminon, Orsay (FR); Laure-Marie Neuburger, Montrouge (FR); Jacques Grassi, Bures sur Yvette (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: Commissariat A L'Energie Atomique, Paris (FR)

(21) Appl. No.: 10/537,000

(22) PCT Filed: Nov. 28, 2003
(86) PCT No.: PCT/FR03/03521

§ 371 Date: Dec. 20, 2005

(30) Foreign Application Priority Data
Nov. 28, 2002 (FR) 0214959

(51) Int. Cl. C12Q 1/68; G01N 33/53; C07D 311/88; C07H 21/04

(52) U.S. Cl. 435/6; 435/7.1; 530/409; 536/25.32; 549/225

(57) ABSTRACT

The invention concerns a method for heterogeneous phase detection of an analyte, the trifunctional reagent used for implementing said method, its use for detecting an analyte, as well as the device for detecting a corresponding analyte

The present invention relates to a method for continuous heterogeneous-phase detection of an analyte, to the trifunctional detecting reagent used during this method, to its use for detecting an analyte, and also to the corresponding device for detecting an analyte.

At the current time, many techniques exist for detecting analytes, and in particular molecules, under laboratory conditions. However, more and more frequently, for health and safety reasons in particular, it is necessary to rapidly detect the presence of compounds that are either compounds of interest (toxins, chemical toxic compounds, hormones, etc.) or products derived from or associated with molecules of interest, or markers of an event or of a particular activity (pesticides, heavy metals, hormones, etc.).

These molecules can be present in very diverse media such as water, air, earth or biological samples, or else in foods.

For optimal protection and/or monitoring, the detection of these molecules should be carried out in real time and continuously. The term “continuous assaying” is intended to mean assaying that makes it possible to permanently monitor the presence or the modifications in concentration of a molecule of interest in a medium.

Among the numerous techniques used to date for detecting these compounds (analytes), a large number use the particularity that certain substances have, of binding specifically to these analytes so as to form complexes. These substances, that will be called “receptor”, may be very diverse in nature: biological (antibodies in whole, fragmented or recombinant form (Fab′, Fab, scFv), receptors, polynucleic acids (DNA or RNA), peptide nucleic acids, lectins, transporter proteins) or chemical (chelates, synthetic receptors). Among these substances, antibodies are most widely used.

Such methods of detection require labeling in order to quantify or detect, by means of a signal, the complexes formed after reaction between the analyte to be detected and the receptor. This labeling can be carried either by the receptor or by a substance (B) corresponding to the analyte or to an analog or to a fragment of the analyte, this labeled molecule often being referred to as a tracer.

Depending on whether the technique does or does not require separation of the formed complexes from the free substances, two major types of detection, referred to as homogeneous-phase or heterogeneous-phase, are distinguished.

Homogeneous-phase assays are carried out in the same medium. They are used when the formation of the complex modifies the signal carried by the receptor or the molecule (B): the formation of the complex can then be directly monitored by measuring the signal. These assays do not require a separation step, allowing them to be more readily automated.

Heterogeneous-phase assays involve, after reaction between the analyte and the receptor, a step consisting of separating the non-complexed tracer from that engaged in the complexes. This separation is often carried out using two media or phases: for example, a solid phase and a liquid phase.

Based on these basic principles, many assaying methods have been described that differ in particular according to the nature of the labeling used, that may, for example, be enzymatic, radioactive or luminescent (Grassi J. et al., Handbook of Experimental Pharmacology, Ed. Springer-Verlag, Berlin, 1987, 87, Chapter 5; Pelizzola D. et al., Q J Nucl. Med., 1995, 39(4) 251-263).

Among these, labeling with a luminescent compound has the advantage of obtaining a localized signal that does not require the presence of other reagents, as is the case for enzymatic labeling. This type of labeling also makes it possible to use a phenomenon such as energy transfer, that can be carried out according to various mechanisms: resonance energy transfer, radioactive energy transfer (the acceptor absorbs the light emitted by the donor), electron transfer (Matko J. et al., Biochemistry, 1992, 31, 703-711; Nikoobakht B. et al., Photochemistry and Photobiology, 2002, 75, 591-597).

This energy transfer, between a luminescent “donor” compound (D) and an “acceptor” compound (A) that may or may not be luminescent, and that is dependent on the distance between A and D, has been used for carrying out many assays. D and A, which are coupled to the acceptor or the analyte, are chosen such that the energy transfer takes place only when the receptor/analyte complex is formed. This phenomenon is reflected by a decrease or extinction of the luminescence of D and an emission of luminescence from A if the latter is luminescent, when D is excited. In these assays, either the variation in luminescence of A, or the variation in luminescence of D is measured, the nature of A and of D being variable.

In this way, various authors have already proposed methods for detection of an analyte by measuring the luminescence of A:

the method described, for example, by R. Arai et al. (Protein Engineering, 2000, 13(5), 369-396) uses, as donor and acceptor, two fluorescent proteins. These two proteins are produced by molecular biology, each one forming a chimeric protein with either the variable region of the heavy chain or the variable region of the light chain of the same antibody. The presence of the molecule against which the antibody is directed induces the formation of a complex with the two chimeric proteins. The distance between the two fluorescent proteins is then compatible with an energy transfer; the excitation of D will therefore induce fluorescence from A, which will be measured;

according to International application WO 96/42016, it is possible to use, as donor, a complex of rare earth metals (europium, terbium) with a chelate, a cryptate or a macrocycle and, as acceptor, a fluorescent protein. A and D can be either coupled to two receptors that can complex simultaneously on the analyte, and the presence of the latter is then reflected by an emission of luminescence from A when D is excited, or A is coupled to a receptor and D to the analyte and the presence of the analyte will induce competition, with the analyte coupled to D, for complexation with the receptor (competitive assay), the luminescence of A then becoming inversely proportional to the amount of the analyte added. This type of assay is called a time-resolved luminescence assay.

Other authors have also proposed methods for detection of an analyte by measuring the luminescence of D. These assays use the ability of a compound (A) to decrease or suppress the luminescence of another compound (D) when said compounds are sufficiently close (“quench”). The range of molecules A that can be used is therefore broader and thus includes non-luminescence compounds such as heavy metals, heavy atoms, chemical molecules, for instance methyl red, nanoparticles such as those sold under the name Nanogold® by the company Nanoprobes (USA), or alternatively the molecules sold under the names DABCYL® (Eurogentec, Belgium), QSY Dyes (Molecular Probes Inc., USA), ElleQuencher® (Oswell/Eurogentec) or Black Hole Quenchers® (Biosearch Technologies Inc., USA).

By way of examples:

U.S. Pat. No. 3,996,345 describes a method using pairs of fluorescent/“quencher” molecules in assays that use antibodies as receptors;

the method proposed by M. Adamczyk et al. (Organic Letters, 2001, 3, 1797-1800) uses a bioluminescent protein (Aequorine®) as donor and QSY-7 or Dabcyl® as acceptor or “quencher”. D is coupled to a biotin molecule and A is coupled to an avidin molecule. The formation of the biotin/avidin complex causes a decrease in the bioluminescence of D, the presence of free biotin or biotin coupled to a protein will induce competition, with the biotin coupled to D, for complexation with avidin, and the bioluminescence of D will then be proportional to the amount of biotin added;

M. Lee et al. (J. Agr. Food Chem., 1999, 47, 2766-2770) and Schobel et al. (Bioconjugate Chem., 1999, 10, 1107-1114) describe the use of two fluorescent molecules as donor and “quencher”. The donor is coupled to an antibody and the acceptor is coupled to the analyte. As above, the formation of the antibody-D/analyte-A complex causes a decrease in the fluorescence of D, which is restored by the presence of the analyte in the medium;

B. Dubertret et al. (Nature Biotechnology, 2001, 19, 365-370) and Bonnet et al. (Proc. Natl. Acad. Sci., 1999, 96, 6171-6176) describe the use of a polynucleic acid (“beacon”) that has the particularity of hybridizing with itself by folding on itself. According to the method described in that article, a fluorophore compound (D) is attached to the 3′ end of the polynucleic acid and an acceptor (A: nanoparticle or Dabcyl®) is attached to the 5′ end. Since the two ends of the “beacon” are close, the fluorescence of D is decreased by A. The presence of a polynucleic acid (analyte) that can hybridize with a region of the “beacon” will induce linearization of the latter and therefore an increase in the distance between D and A, and will thus decrease the quenching of the fluorescence of D by A;

U.S. Pat. No. 5,279,943 proposes a method for detection of an analyte in which heavy atoms are used as “quenchers” of the luminescence;

U.S. Pat. No. 5,229,302 describes a method for detection of an analyte in a medium, in which a chemical compound is coupled to a ligand or to an analog thereof, which, when the antibody/ligand complex is formed, quenches the fluorescence emitted by the antibody when there is excitation at 280 nm. The presence of the analyte in the medium will induce competition for complexation with the antibody and thus restore the fluorescence of the antibody;

C. T. Chen et al. (Science, 1998, 279, 851-852) have described a method for detection of an analyte in a medium by means of a synthetic receptor to which a fluorophore and a quencher are attached. In the absence of analyte, the quencher is sufficiently close to the fluorophore to decrease its fluorescence. Complexation of the analyte on the receptor will increase the distance between the quencher and the fluorophore and thus increase the fluorescence of the latter.

It should be noted that all the assays using energy transfer described above are homogeneous-phase assays.

Now, in the context of continuous detection of an analyte, flow assays appear to be particularly suitable since they allow a permanent supply of sample.

Thus, several types of flow assay have already been described:

several authors have in particular proposed methods for assaying analytes using an antibody immobilized on the walls of a capillary, this antibody being saturated with a labeled analyte or with a labeled analog thereof. Passage of the analyte into the capillary induces competition with the labeled analyte for binding with the antibody. Thus, a certain amount of the labeled analyte will be released and detected as it exits the capillary (see in particular U.S. Pat. Nos. 5,183,740 and 6,323,041 and Sheikh et al., Biosensor & Bioelectronics, 2001, 16, 647-652);

conversely, C. Barzen et al. (Biosensor & Bioelectronics, 2002, 17, 289-295) have described a method for detection of an analyte in which they use a solid phase on which the analyte or an analog of the analyte to be detected is immobilized. The sample possibly containing the analyte to be detected is preincubated with a labeled anti-analyte antibody. When this solution passes over the solid phase, the non-complexed antibodies will attach to the solid phase via the immobilized analyte. Thus, the fluorescent signal measured on the solid phase is inversely proportional to the amount of analyte present in the sample;

T. E. Plowman et al. (Anal. Chem., 1999, 71, 4344-4352) have developed an assay method for simultaneously assaying various analytes on the same solid phase. According to this method, antibodies that recognize various analytes are immobilized on a solid phase at different places. A solution, containing the sample (with the various analytes) and various anti-analyte antibodies labeled with a fluorophore, is deposited onto the solid phase. Finally, the fluorescence that is derived from the complexation of the capture antibodies, of the analyte and of the trace antibodies, corresponding to the various antibody immobilization sites, is measured. As for the previous assay, the need to mix the analyte with a labeled antibody makes it difficult to apply this method to a continuous assay since this would involve a permanent supply of reagent;

F. S. Ligler et al. (Environmental Science & Technology, 1998, 32, 2461-2466) have developed a biosensor for detecting molecules present in the air. This biosensor consists of a fiber to which avidin molecules are coupled. Biotinylated anti-analyte antibodies are immobilized on this fiber via the avidin/biotin complex. Detection is carried out by passing the sample (molecules present in the air, solubilized in a buffer) over this fiber, followed by anti-analyte antibodies labeled with a fluorophore. Finally, the fluorescence, derived from the complexation of the capture antibodies, of the analyte and of the tracer antibodies, is measured. However, the various steps required to carry out this method and the supply of labeled antibodies, which should be permanent, does not allow a real continuous detection.

It therefore emerges from the description of the various known methods of detection of the prior art that all the flow assays available to date are heterogeneous-phase (solid phase/liquid phase) assays. In fact, homogeneous-phase flow assays would require the continuous supply of labeled analytes and/or receptors, which would be expensive and more difficult to implement.

Finally, the use of capillaries for carrying out heterogeneous-phase analyte assays has also already been proposed, in particular in U.S. Pat. No. 5,976,896, according to a method that can be carried out in accordance with several variants:

according to a first variant of this method, a first receptor for the analyte to be detected is immobilized at the inner surface of the capillary. The sample containing the analyte is preincubated with a second receptor (that does not have the same analyte-binding site as the first receptor attached to the capillary) labeled with a fluorescent compound. This solution, after reaction, is introduced into the capillary; the analyte/labeled receptor complex will then attach to the surface of the capillary by binding to the first receptor. After washing, the fluorescent signal, which is proportional to the amount of analyte in the sample, is measured;

according to a second variant of this method, an analog of the analyte is immobilized at the inner surface of the capillary. The sample containing the analyte to be detected, preincubated beforehand with a receptor for the analyte labeled with a fluorescent compound, is introduced into the capillary. Competition for binding to the labeled receptor will take place between the analyte attached to the surface of the capillary and the analyte possibly present in the sample. After washing, the fluorescent signal, which is inversely proportional to the amount of analyte present in the sample, is measured;

according to a third variant of this method, a receptor for the analyte to be detected is immobilized at the inner surface of the capillary. The sample containing the analyte, mixed beforehand with a labeled analyte, or an analog of this analyte, labeled with a fluorescent compound, is introduced into the capillary. Competition for binding to the receptor attached to the surface of the capillary will then take place between the labeled compound and the analyte of the sample. After washing, the fluorescent signal, which is inversely proportional to the amount of analyte in the sample, is measured.

The various steps in these assays, (incubations, washes) and the need to mix the analyte with a labeled compound makes it difficult to apply this assay to a continuous assay since this will involve a permanent supply of reagent.

Moreover, all the methods for heterogeneous-phase detection of analytes described above most commonly require a distinct step of regeneration of the solid phase to be carried out before it is possible to perform any further detection of the analyte in a new sample. This limiting step of regeneration of the solid phase consequently prohibits the application of these various methods to the continuous detection of an analyte in a medium.

Therefore, in order to remedy all these drawbacks and to provide in particular a method for heterogeneous-phase detection of an analyte, which is simple to implement, which can be automated, and during which the solid-phase regeneration step can be carried out simultaneously with the step for detection of the analyte per se without interrupting the latter, the inventors have developed that which is the subject of the present invention.

The inventors also gave themselves the aim of providing a novel reagent that can be used in methods for heterogeneous-phase detection of analytes that make it possible to avoid any incubation, prior to the detection step per se, of the analyte with a labeled antibody or a labeled analyte.

A first subject of the present invention is therefore a method for detection of an analyte a in a fluid sample, characterized in that it comprises the following steps:

1) saturating a solid support comprising, on at least part of its surface, at least one trifunctional reagent (tripod Y) comprising the following three functional poles:

i) a luminescent group (L),

ii) a molecule (B) chosen from the analyte a, an analog of the analyte a or a fragment of the analyte a; and

iii) a function that provides attachment of said trifunctional reagent to the surface of said solid support,

with a receptor for the analyte a, said receptor being labeled with a compound (Q) (receptor-Q) that quenches the luminescence of the group L, so as to form a complex C between said molecule (B) and said receptor-Q;

2) bringing the solid support obtained in step 1) into contact with a fluid sample that may contain the analyte a to be detected;

3) measuring the intensity of the signal emitted by the group L, which is proportional to the amount of analyte a present in the fluid sample; and

4) regenerating the solid support by bringing said solid support into contact with the receptor-Q.

According to this method, step 1) allows complexation of the molecule (B) with the receptor-Q. At the end of this first step, the luminescence of L is decreased or suppressed. In step 2), bringing the sample into contact with the surface of the solid support will induce, when said sample contains the analyte a, competition between the analyte a and the molecule (B) for the formation of the complex with the receptor-Q. The binding of the analyte a to the receptor-Q will result in the receptor-Q being eliminated from the surface of the solid support and in the luminescence emitted by the compound (L) present on the tripod Y being restored. The intensity of the signal measured in step 3) is then proportional to the amount of analyte a present in the sample to be analyzed. The regeneration step 4) will again bring about complexation of the receptor-Q on the tripod Y and thus suppression of the luminescence of L, so as to allow further detection of the analyte a in a new sample.

The principle of the method for detection of an analyte a in accordance with the invention is represented diagrammatically in scheme A below: 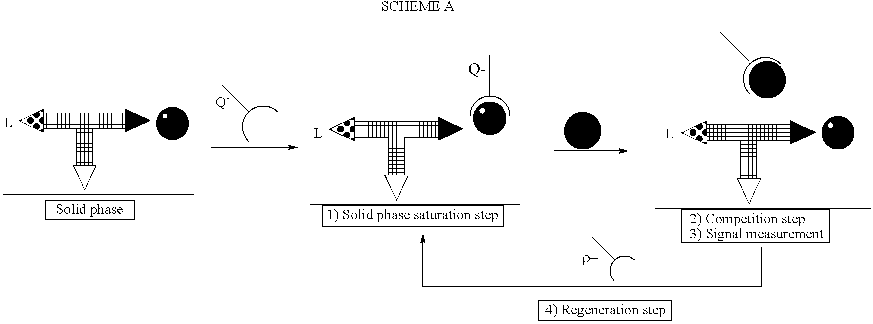
The method of detection in accordance with the invention has a large number of advantages:

1) due in particular to the specific structural conformation of the tripod Y used during the method, the step consisting of regeneration of the solid phase can be carried out very readily, without any alteration of its properties. In fact, in all the flow assays described above, the signal is measured after formation of a complex bound to the solid phase. Consequently, and even though some of the assaying methods described by the prior state of the art allow several successive assays (Sheikh et al. and U.S. Pat. Nos. 5,183,740 and 6,323,041; mentioned above), they are however limited in number and require a solid support regeneration step that is often long and restricting, which results in the dissociation of the complex formed. In addition, the drastic conditions for carrying out these regeneration steps (passing over acid or basic solutions) mean that the detection method must be stopped, thus prohibiting any application of these methods to continuous assays of a given analyte.

On the other hand, according to the method of detection in accordance with the invention, the presence of the analyte a in the sample brings about dissociation of the receptor-Q and of the molecule (B); the regeneration step therefore consists quite simply in reforming this complex by adding receptor-Q. This regeneration step does not therefore involve the use of acid or basic solutions that may impair the properties of the molecules of the solid phase or an exchange reaction between two molecules at the receptor binding site, the kinetics of which are longer than the reaction to form a complex.

2) the signal is measured in the region on which the tripod Y was immobilized, which makes it possible to obtain a localized signal, unlike the assays developed according to the prior art, in particular by Sheikh et al., and also in U.S. Pat. No. 5,183,740 and 6,323,041 mentioned above, in which the signal, bound to molecules in solution, is measured at the outlet of a capillary.

3) since the signal is localized, several molecules may be detected simultaneously on the same solid support by attaching, to distinct and known zones thereof, several types of tripods Y that differ from one another through the nature of the molecule (B) that they comprise.

4) the signal measured corresponds to all the molecules of analyte a that have been in contact with the solid support between two regenerations. This particularity of the method in accordance with the invention allows permanent monitoring to be obtained while at the same time taking measurements that are spaced out over time.

5) this assay format is applicable to all molecules since it does not require the simultaneous binding of two receptors to the analyte, as is sometimes necessary according to the assay methods previously known, for instance in the method described by T. E. Plowman et al., mentioned above, and which require the analyte to be of sufficient size.

6) the presence of analyte a in the sample results in the appearance of a signal, unlike most of the competition assays known from the prior art, the appearance of a signal allowing easier detection.

7) finally, since the detection system uses the energy transfer phenomenon, it also makes it possible to detect and to quantify the presence of an analyte a by means of the variation in luminescence of the compound Q if it is fluorescent or the variation in the apparent time for decrease in luminescence of the compound (L).

According to a particularly preferred embodiment of the method in accordance with the invention, step 3) consisting in measuring the intensity of the signal emitted and step 4) consisting in regenerating the surface of the solid support are carried out continuously.

According to the invention, the solid support is preferably chosen from materials comprising at their surface, naturally or after modification, functions capable of forming a bond of covalent nature with a complementary function of the tripod Y. These functions may in particular be hydroxyl, amine, sulfhydryl or carboxyl functions.

Among such materials, mention may in particular be made of glasses, plastics (for example polystyrene), ceramics (preferably of oxide type), metals (for example aluminum or gold) and metalloids (such as oxidized silicon).

Such supports can in particular be in the form of tubes, capillaries, plates such as microplates, or beads, or in any other form suitable for implementing the method in accordance with the invention.

The fluid sample that may or may not contain the analyte a to be detected may be of diverse nature or origin, such as, for example, water, a liquid biological medium, or alternatively a liquid containing dissolved gaseous molecules or molecules originating from solid samples.

The intensity of the signal emitted during step 3) can be measured by means of a luminescence detector such as, for example, a fluorimeter.

According to the method in accordance with the invention, the complex C formed at the end of the saturation step 1) is preferably chosen from the complexes of formula (I) below:

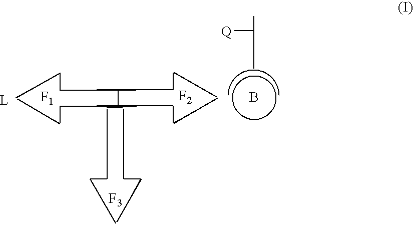

in which:

the arrows represent the structure of the backbone of the tripod Y, which is a linker arm consisting of a peptide, nucleotide or glucoside chain or of a saturated or unsaturated, linear or branched hydrocarbon-based chain; said chains being optionally substituted, interrupted and/or ended with one or more hetero atoms, such as N, O or S, and/or with one or more amino acids, and comprising three reactive chemical functions F₁, F₂ and F₃;

L represents a luminescent group covalently bonded to the tripod Y by means of the reactive chemical function F₁;

B represents an analyte a, a structural analog of an analyte a or a fragment of an analyte a to which is noncovalently and reversibly attached a receptor specific for the analyte a, said receptor being labeled with a compound Q; the molecule (B) being covalently bonded to the tripod Y by means of the reactive chemical function F₂;

Q represents a compound that quenches the luminescence of the group L;

F₃ represents a reactive chemical function that can allow the attachment of the tripod Y to the surface of the solid support.

According to the invention, and independently of one another, the functions F₁, F₂ and F₃ provide:

i) either a direct linkage via a corresponding chemical function present on the luminescent compound, the molecule (B) or the solid phase;

ii) or an indirect linkage. In this second case, the linkage may be carried out by coupling, to at least one of the functions F₁, F₂ and/or F₃, a molecule Ml capable of forming a complex with a molecule M₂ attached beforehand to at least part of the surface of the solid phase, to the molecule (B) and/or to the luminescent group. By way of example, it is in particular possible to couple biotin or streptavidin (or neutravidin or avidin) to one of the functions F₁, F₂ and F₃ and, respectively, streptavidin (or neutravidin or avidin) or biotin to the solid phase, to the molecule (B) and/or to the luminescent compound. According to another variant of the method in accordance with the invention, this indirect linkage can also take place via a molecule M₃, coupled beforehand to the solid phase with one or more luminescent compounds or molecules (B) and which will be coupled to the function F₁, F₂ or F₃. By way of example of molecules M₃, use may in particular be made of proteins such as albumin and polylysine, nucleotides, sugars or alternatively other synthetic derivatives.

Preferably, F₁, F₂ and F₃, which may be identical or different, are chosen from the following functions: thiols; amines; alcohols; acid functions such as carboxylic acid functions; esters such as activated esters, for instance succinimidyl esters and anhydrides; isothiocyanates; isocyanates; acylazides; sulfonyl chlorides; aldehydes; glyoxals; epoxides; oxiranes; carbonates; imidoesters; carbodiimides; maleimides; nitriles; aziridines; acryloyl; halogenated derivatives; disulfide groups; phosphorus-containing groups such as, for example, phosphates, phosphonates, phosphines and phosphites; diazo; carbonyldiimidazole; hydrazides; arylazides; hydrazines; diazirines; magnesium compounds; lithium compounds; cuprates; zinc compounds and unsaturated systems.

Among these various functions and groups, mention may in particular be made of amine functions such as those of formulae R—NH₂, R—NH—, (R)₃—N, R—NH—OR and NH₂—OR; alcohol functions R—OH; and halogenated groups of formula R—X with X representing a halogen atom such as chlorine, iodine, bromine or fluorine; it being understood that, in said formulae, R represents an alkyl, preferably C₁-C₁₅ alkyl, aryl, vinyl or allyl radical.

According to the invention, the term “aryl group” is intended to mean any aromatic group having one or more benzene, naphthalene or anthracene rings, said rings optionally containing one or more hetero atoms, such as O, N or S, and being optionally substituted with one or more groups chosen from halogen atoms, and C₁-C₄-alkyl, amino, amino(C₁-C₄)alkyl, (C₁-C₄) alkyleneamino (C₁-C₄) alkyl, di (C₁-C₄) alkylamino-(C₁-C₄)alkyl, nitro, C₁-C₄ alkyleneamino or C₂-C₄ alkenyleneamino radicals.

Among such aryl groups, mention may in particular be made of benzyl, phenyl, cresyl, toluyl, pyridine, pyrimidine and pyrazine groups.

According to the invention, the term “luminescent group” is intended to mean any substance which, when it is excited at a given wavelength or by a given chemical compound, is capable of emitting a photon, for example a fluorophore or rare earth metal.

Among such luminescent groups, mention may in particular be made of fluorescein (sodium fluoresceinate) and its derivatives such as fluorescein isothiocyanate (FITC); rhodamine and its derivatives such as tetramethyl rhodamine isothiocyanate (TRITC); diaminidophenyl indo (DAPI); acridine; fluorescent dyes with reactive amines, such as the succinimidyl ester of 6-((7-amino-4-methylcoumarin-3-acetyl)amino)hexanoic acid (AMCA); the fluorescent dyes sold under the brand names Bodipy®, such as Bodipy® FR—Br₂, Bodipy® R6G, Bodipy® TMR, Bodipy® TR and Bodipy® 530/550 (excitation wavelength/emission wavelength, in mm), 558/568, 564/570, 576/589, 581/591, 630/650 and 650/665 sold by the company Bio-Rad Inc. (USA), the dyes Cascade Blue (Trilink BioTechnologies (USA), Cy2, Cy3, Cy3.5, Cy5, Cy5.5 and Cy7 (Bio-Rad Inc., USA), Dabcyl® and Edans® (Eurogentec, BE); eosin; erythrosine; 6-Fam and Texas Red.

According to the invention, the term “receptor” is intended to mean any substance capable of forming a noncovalent and reversible linkage (complex) with the analyte, an analog of the analyte or a fragment of the latter. This receptor is of course chosen according to the nature of the molecule (B) of the tripod Y.

These receptors can in particular be chosen from compounds that are biological in nature (antibodies in whole, fragmented or recombinant form (Fab′, Fab, scFv), receptors, polynucleic acids (DNA or RNA), peptide nucleic acids, lectins or alternatively transporter proteins) and compounds that are chemical in nature, such as, for example, specific synthetic receptors and chelates.

Among such receptors, mention may, for example, be made of monoclonal anti-substance P, anti-prion protein or anti-angiotensin II antibodies, polyhistidine, the nitrilo-triacetic acid-nickel (NTA-nickel) system, and complementary nucleotide probes.

According to a preferred variant of the method of detection in accordance with the invention, said receptor exhibits greater affinity for the analyte a than for the molecule (B).

According to the invention, the term “quenching compound” (Q) is intended to mean any molecule that allows a decrease in or the disappearance of the luminescence of the luminescent compound (L) when the receptor is complexed with the molecule (B). This compound, that may be diverse in nature, may in particular be a chemical compound (luminescent or nonluminescent), a heavy atom or a nanoparticle.

Among such compounds (Q), mention may in particular be made of fluorescent compounds such as those mentioned above for the groups L, rhodamine and its derivatives such as tetramethyl rhodamine (TMR), nonfluorescent molecules such as the compounds sold under the brand names Black Hole Quencher® 1, 2 and 3 (Biosearch Technologies), Nanogold Particules® (Nanoprobes), Eclipse Dark Quencher® (Epoch Bioscience), Elle Quencher® (Oswell), malachite green, and the dyes QSY® 7, QSY® 9 and QSY® 21 (Molecular Probes).

When the molecule (B) is a peptide or an oligonucleotide, then the tripod Y used according to the method in accordance with the invention can be prepared by carrying out a peptide or oligonucleotide synthesis during which at least one amino acid (or modified nucleotide) comprising a function (F₁) and another amino acid (or modified nucleotide) comprising a function (F₃) is added to said molecule (B), F₁ and F₃ having the same meanings as those indicated above. In this case, F₁ and F₃ also provide the linkage respectively with the luminescent compound (L) and the surface of the solid phase. Just as above, the amino acids (or nucleotides) comprising the chemical functions F₁ and F₃ can also be replaced with an amino acid (or nucleotide) coupled to a biotin (for example, the product 9-fluorenylmethoxycarbonyl (Fmoc)-lysine(biotin)-OH, sold by the company Calbiochem-Novabiochem AG). In this case, this amino acid (or nucleotide) will provide the linkage with the surface of the solid phase or with the luminescent compound (L) to which streptavidin (or neutravidin or avidin) will have been coupled beforehand, so as to form an indirect linkage.

Among the complexes C of formula (I) in accordance with the invention, mention may in particular be made of the compounds in which:

i) (B) is chosen from peptides, proteins, oligonucleotides, sugars and peptide nucleic acids,

ii) L is fluorescein, and

iii) the backbone of the tripod Y is chosen from the structures Y₁ to Y₃ below:

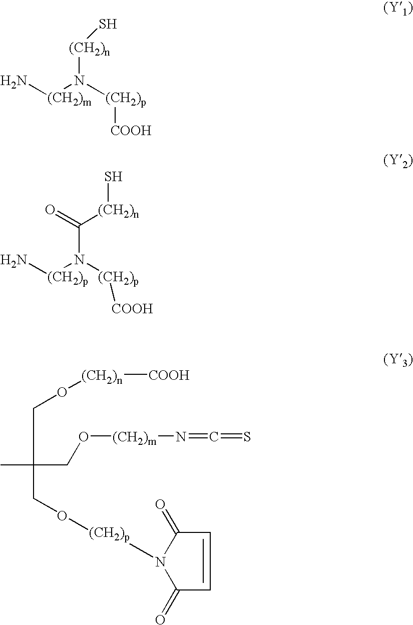

in which n, m and p, which may be identical or different, are integers between 1 and 20 inclusive.

Among the structures Y₁ to Y₃, the compounds of formulae (Y′₁) to (Y′₃) below are particularly preferred:

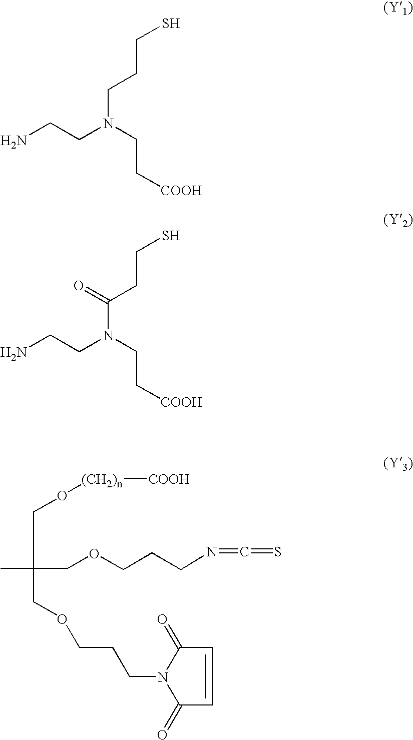

The tripods Y used in the method in accordance with the invention can be prepared by analogy according to the methods of organic and peptide synthesis conventionally used and well known to those skilled in the art.

The complexes C of the formula (I) as described above are compounds that are novel in themselves, which, in this respect, constitutes another subject of the invention.

These complexes C of the formula (I) can be prepared by complexing a tripod Y in accordance with the invention with a receptor-Q, according to the methods conventionally used in the state of the art.

A subject of the invention is also the use of at least one complex C of formula (I) in a method for continuous heterogeneous-phase detection of an analyte a in a fluid sample.

Finally, a subject of the invention is a device for continuous heterogeneous-phase detection of at least one analyte a in a fluid sample, said device being characterized in that a fluid sample to be analyzed is integrated into a medium forming a stream that flows over at least one solid support at the surface of which is attached at least one tripod Y in accordance with the invention as described above and specific for the analyte a to be detected, a luminescence detector placed opposite the solid support is coupled to a valve control that is controlled by a threshold of intensity of signal emitted by the detector and which triggers, for a given period of time, the opening of a reservoir containing a receptor-Q capable of forming a complex with the tripod Y, this reservoir being linked to the support via a feedback loop which comes in upstream of the solid support to which the tripod Y is attached, in order to saturate and/or regenerate the latter with receptor-Q by passage in the stream and complexation on the tripod Y.

According to specific embodiments:

the luminescence intensity values are monitored and secondarily translated into an amount of analyte a by a calculation system coupled to the luminescence detector;

an event marker, for instance an alarm, is placed in the feedback loop in order to signal a variation in intensity of the signal above a predetermined value;

the solid support is a capillary coupled to the environment containing the sample to be analyzed, the coupling being carried out either by means of a round-bottomed capture flask in which the sample sparges in a medium corresponding to that of the flow stream, or by means of a flexible pipe;

the stream is entrained by means of the low pressure produced by a pump, a piston, or equivalent.

These devices can in particular be used for detecting the presence of an analyte a in a natural medium, for instance especially in lakes and rivers, or in industrial media such as swimming pools, factories, purification plants, ventilation or air-conditioning systems, etc. Where appropriate, they can be equipped with a round-bottomed capture flask and with a sparging system for collecting samples in gaseous form, such as air, and for solubilizing the constituents to be detected that they contain.

Besides the above provisions, the invention also comprises other provisions that will emerge from the following description, which makes reference to examples of synthesis of backbones of tripods Y, to an example of synthesis of a complex of formula (I) and to two examples of detection of substance P in a fluid sample, and also to the attached FIGS. 1 to 8 in which:

FIG. 1 illustrates a general view of a nonlimiting example of a heterogeneous-phase detection device according to the invention, for measuring the presence of an analyte a in a water purification plant tank. According to this device, a piece of flexible tubing 1 connected to a tank outlet is mounted on a capillary tube 3. This tube serves as a support for the attachment of two types of tripod according to the invention, namely Ya and Yb, allowing the specific detection of two types of analytes. The liquid sample originating from the tank flows as a continuous stream (arrows Fa) in the capillary 3 by means of the action of a peristaltic pump 5, and is then evacuated. In the vicinity of the capillary and opposite each area of the capillary to which the tripods Ya and Yb are attached, the placement of a fluorimeter, 7a and 7b, for detecting fluorescence is envisioned. Each detector is connected to a reservoir of receptors-Q, 9a and 9b, respectively specific for the tripods Ya and Yb. The reservoirs are connected, via side channels 11a and 11b and a common channel 11c, to the capillary 3, upstream of the areas to which the tripods Ya and Yb are attached, relative to the direction of flow of the stream. The common channel is welded perpendicular to the capillary 3 and all the channels form feedback loops. Each detector is coupled to a valve control, respectively 13a and 13b, for opening the corresponding reservoir, respectively 9a and 9b, and so as to allow its content to pour out into the capillary 3 (arrows Fb) and then to complex on the corresponding specific tripod, Ya or Yb, via the channels 11a, 11b and 11c, in order to regenerate this tripod. In this example, the regeneration can be carried out continuously, the threshold for triggering this regeneration operation corresponding to the minimum variation in luminescence that can be detected according to the sensitivity of the detector. An event marker 15a (respectively 15b) is mounted in each feedback loop. In operating mode, and after calibration, the intensity of the fluorescent signal, Ia and Ib, emitted by the tripods Ya and Yb is measured by the fluorimeter, which makes it possible to calculate, using an attached calculating device (not represented), the average concentration of analyte under examination in the sample. The valve opening control 13a (respectively 13b) is controlled by the intensity of fluorescent signal measured by the corresponding detector. Opening of the corresponding valve is then triggered for the period of time corresponding to the regeneration of the tripods concerned. If the analyte a is present in the sample during the regeneration, this results in an increase in the duration thereof (some of the receptor-Q complexing with the analyte a). The duration of the regeneration can be determined by the time necessary for the fluorescence to return to the basal level; if this is greater than a predetermined value, an event marker may be triggered. When the intensity of the signal varies by an amount greater than a predetermined value, for a given time interval, the event marker 15a (respectively 15b) signals this fact by means of a visual and/or sound warning 16. So if the fluorescent signal corresponds to all the analytes a that have passed through the capillary between two regenerations, the fluorescence can also be read at regular time intervals. In other examples, when the sample is a gas, the medium used and the means for creating a stream are adapted by those skilled in the art;

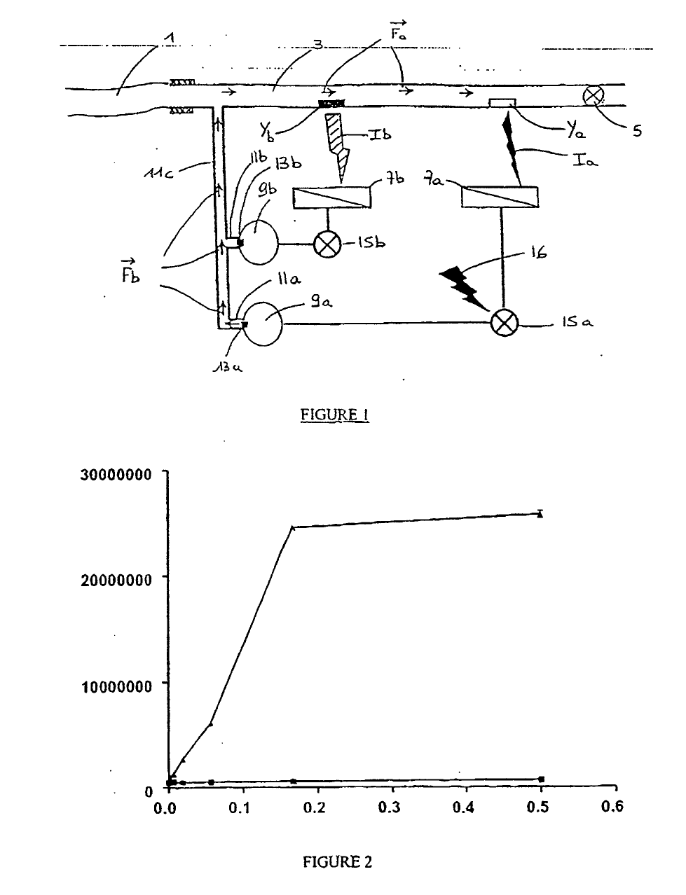

FIG. 2 represents the fluorescence measured, in arbitrary units, after immobilization on a microtitration plate, via neutravidin, of a tripod Y comprising fluorescein as compound (L) and a substance P analog as molecule (B), as a function of the concentration of tripod in μM;

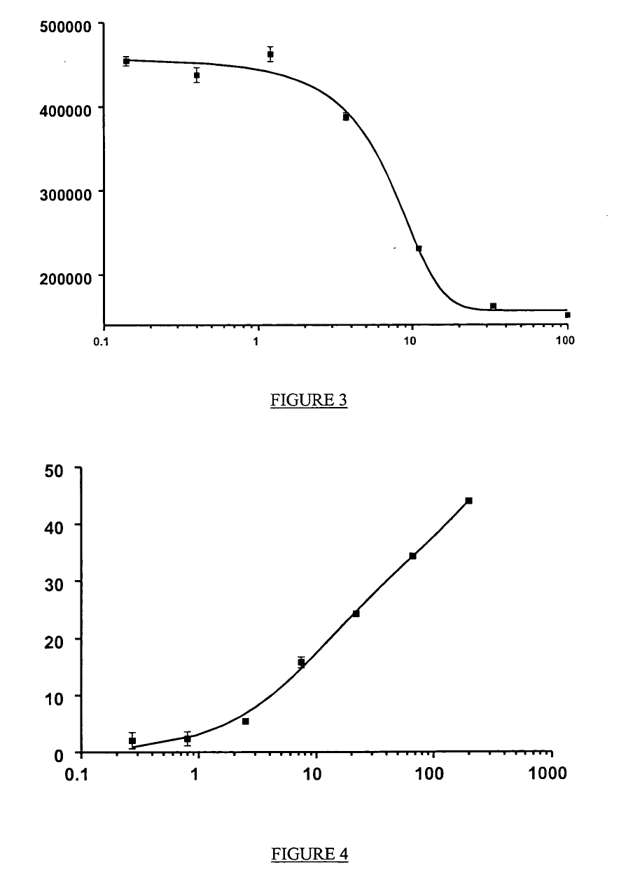

FIG. 3 represents the curve for decrease in the fluorescence (in arbitrary units) of a tripod comprising fluorescein as compound (L) and a substance P analog as molecule (B), as a function of the amount of tetramethyl rhodamine-labeled anti-substance P monoclonal antibody (mAb SP31-TMR) (in nM) that complexes with the tripod;

FIG. 4 represents the percentage inhibition by substance P of the decrease in fluorescence caused by the mAb SP31-TMR (% ID), expressed as a function of the concentration of substance P (in nM);

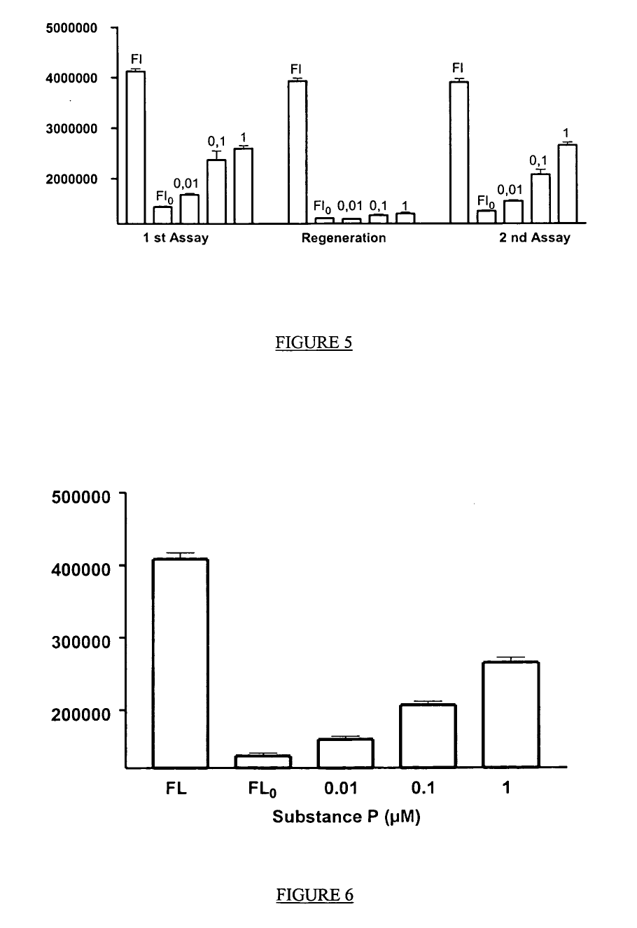

FIG. 5 represents the fluorescence measured (in arbitrary units) after the bringing into contact of a solid support to which a tripod Y in accordance with the invention is attached, said tripod possibly being complexed with the mAb SP31-TMR (well Fl: fluorescence measured in the absence of mAb SP31-TMR and of substance P and well Fl₀: fluorescence measured in the presence of mAb SP31-TMR but in the absence of substance P), with various concentrations of substance P (well Flₓ: fluorescence measured in the presence of mAb SP31-TMR with x=concentration of substance P: 1; 0.1 or 0.01 μM), during a first assay, and then after regeneration of the support and, finally, after the wells have again been brought into contact with the three concentrations of substance P;

FIG. 6 represents the mean fluorescence measured (in arbitrary units) for each of the wells Fl, Fl₀ and Flₓ, after 11 substance P-assay and regeneration cycles;

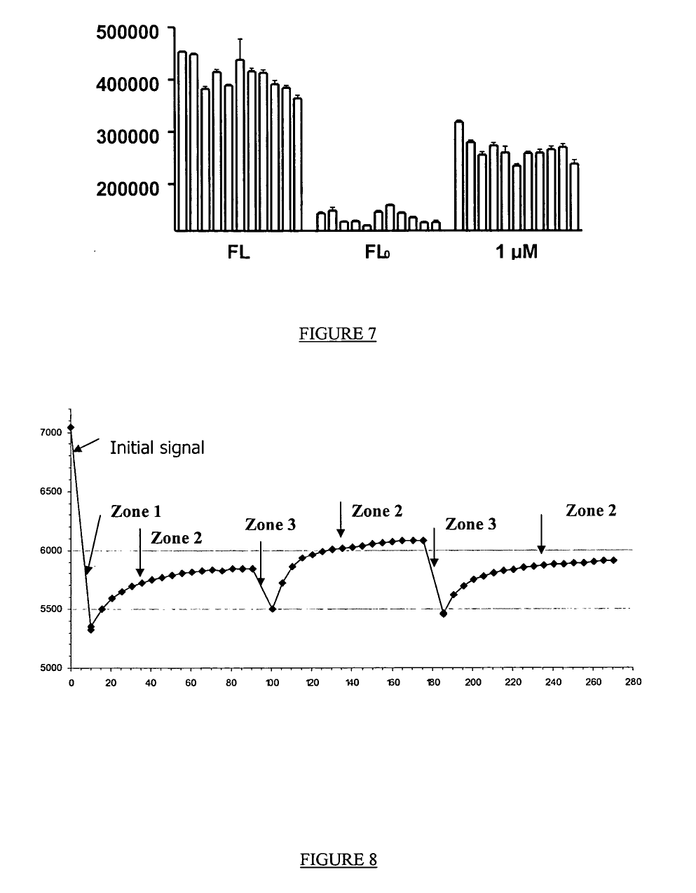

FIG. 7 represents the fluorescence (in arbitrary units) measured for each of the 11 assays carried out in wells Fl, Fl₀ and Flₓ, with x=1 μM of substance P;

FIG. 8 represents the change in fluorescence (in arbitrary units) as a function of time, of a capillary functionalized with a tripod Y in accordance with the invention, brought into contact with a labeled monoclonal antibody mAb SP31 (zone 1), and then with substance P (zone 2), and regenerated by bringing the tripod attached to the capillary into contact with the labeled monoclonal antibody mAb SP31 (zone 3), the last two steps being repeated three times.

### EXAMPLE 1

### Preparation of a Backbone of Formula (Y′₁) of a Tripod Y In Accordance with the Invention

One equivalent of 1,2-ethyldiamine is reacted with two equivalents of (Boc)₂O to give bis(tert-butyloxycarbonylamino)-1,2-ethyl, which is subsequently reacted with one equivalent of phenyl 3-bromopropanoate in the presence of sodium hydride to give the compound of formula (Y′1a) below:

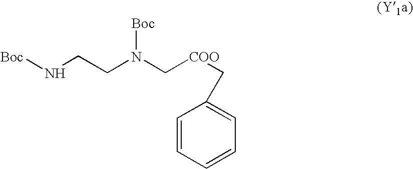

The compound Y′1a is deprotected in a trifluoroacetic medium and then the deprotected compound obtained is condensed in the presence of triethylamine (TEA) and of one equivalent of Z chloride (with Z=carbobenzyloxy), so as to obtain a compound of formula (Y′1b) below:

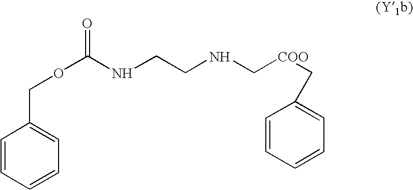

The compound of formula (Y′1b) then gives, in the presence of S-(3-chloropropyl)ethanethioate and of TEA, the compound of formula (Y′1c) below:

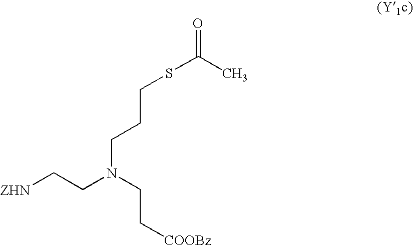

in which Z has the same meaning as that indicated for the compound of formula (Y′1b) above and Bz denotes a benzyl group.

The acid and primary amine functions of the compound of formula (Y′1c) are deprotected in the presence of palladium-on-charcoal and the thiol function is deprotected by the action of hydroxylamine, to give the compound of formula (Y′1).

### EXAMPLE 2

### Preparation of a Backbone of Formula (Y′₂) of a Tripod Y in Accordance with the Invention

3-[(2-Oxopropyl)thio]propanoic acid, prepared from 3-bromopropanoic acid and from ethanethioic acid, is reacted with the compound of formula (Y′1b) obtained above in example 1, to give the compound of formula (Y′2a) below:

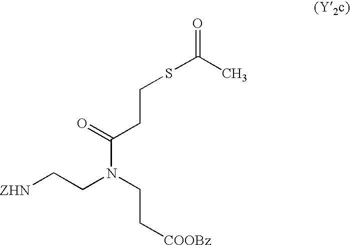

in which Z denotes a carbobenzyloxy group and Bz denotes a benzyl group.

The acid and primary amine functions of the compound of formula (Y′2a) above are deprotected in the presence of palladium-on-charcoal and the thiol function is deprotected by the action of hydroxylamine, to give the compound of formula (Y′2) described above.

### EXAMPLE 3

### Preparation of a Backbone of Formula (Y′₃) of a Tripod Y in Accordance with the Invention

One equivalent of 2-(hydroxymethyl)-2-methylpropane-1,3-diol and one equivalent of ethyl 3-bromopropanoate are reacted in the presence of sodium hydride, to give ethyl 3-[3-hydroxy-2-(hydroxymethyl)-2-methylpropoxy]propanoate. This compound is subsequently reacted, in the presence of sodium hydride, with one equivalent of tert-butyl 3-bromopropylcarbamate, to give the compound of formula (Y′3a) below:

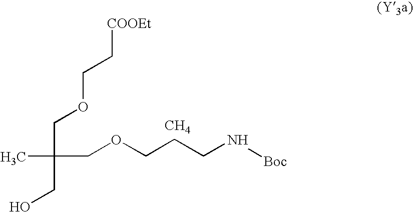

The compound of formula (Y′3a) above is subsequently deprotected in a trifluoroacetic medium and then reacted with dithioxomethane in the presence of sodium hydroxide, and the carboxylic group is then reprotected with an ethanol/sulfuric acid mixture, to give the compound of formula (Y′3b) below:

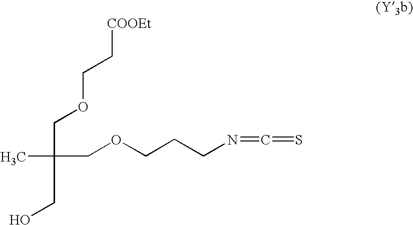

The compound of formula (Y′3b) above is subsequently reacted with 1-(3-iodopropyl)-1H-pyrrole-2,5-dione in the presence of sodium hydride, to give the compound of formula (Y′3c) below:

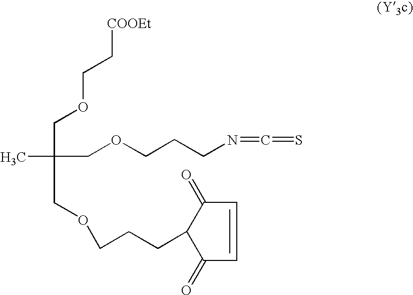

which, in the presence of sodium hydroxide, gives the compound (Y′3).

### EXAMPLE 4

### Preparation of a Tripod Y in Accordance with the Invention

This example illustrates the preparation of a tripod Y in accordance with the invention comprising:

a substance P analog as molecule (B),

an NH₂ function for attachment of the tripod to the surface of a solid support (F₃),

fluorescein as luminescent compound (L).

1) Preparation of a Substance P Analog

As a matter of interest, the sequence of substance P (molecular weight: MW=1349) is as follows:

Arg-Pro-Lys-Pro-Gln-Gln-Phe-Phe-Gly-Leu-Met-CONH₂
i.e. (SEQ ID No. 1)-CONH₂.

A substance P analog corresponding to the sequence below (MW=1907) is prepared:

Lys-Ser-Ser-Lys(Biotin)-Arg-Pro-Ala-Pro-Gln-Gln-
Phe-Phe-Gly-Ala-Met-CONH₂;
i.e. (SEQ ID No. 2)-CONH₂.

To do this, the coupling of the substance P analog and of the biotin, and also of the NH₂ function, is carried out in the course of a peptide synthesis according to a method known to those skilled in the art, for instance a solid-phase synthesis as described by Merrifield (J. Am. Chem. Soc., 1964, 85, 2149-2154). The substance P analog is a peptide analog of substance P comprising an alanine at position 10 [Ala¹⁰], which shows 13% cross reactivity with substance P for the monoclonal antibody SP31 (O. Déry, Biopolymers, 1996, 39, 67-74), which is used in the assay described below as a receptor. This weak affinity of the monoclonal antibody SP31 (mAb SP31) for this analog compared with substance P will allow better displacement from the antibody attached to the solid phase. On this analog, the lysine at position 3 (Lys3) was also replaced with an alanine in order to eliminate the primary amine present on the side chain. Since Lys3 is not in the recognition site of the mAb SP31, this replacement does not modify the affinity of the antibody for the analog. Moreover, the N-terminal end was modified by the addition of a lysine having a biotin molecule on the side chain (Fmoc-Lys(biotin)-OH), and two serines followed by a lysine having two NH₂ functions, one of them being on its side chain.

After synthesis, the compound obtained is purified by high performance liquid chromatography (HPLC) and then lyophilized.

2) Labeling of the Substance P Analog with Fluorescein (L)

100 μg of the compound obtained above in 1) (5.28×10⁻⁸ mol) are dissolved in 100 μl of 0.1 M borate buffer, pH 9. 30.8 μl (5.28×10⁻⁷ mol) of a solution of fluorescein-NH-succinimide (MW 586.55) (6-(fluorescein-5-(and-6)-carboxamido)hexanoic acid, succinimidyl ester sold under the reference F-6129 by the company Molecular Probes, Inc.) at 10 mg/ml in dimethylformamide (DMF) was subsequently added. After stirring at a temperature of 4° C. for 16 hours, 100 μl of 1 M Tris-HCl buffer, pH 9, are added and the stirring is continued for 16 hours at 4° C. in order to inactivate the residual active ester functions. Finally, 769.2 μl of 0.1 M potassium phosphate buffer, pH 7.4, and 0.01% of sodium azide are added. A solution of a tripod Y in accordance with the invention at 52.8 μM is obtained. This tripod is conserved at a temperature of 4° C.

### EXAMPLE 5

### Detection and Assaying of Substance P in a Sample

1) Preparation of a Receptor-Q: Labeling of the Anti-Substance P Monoclonal Antibody SP31 with Tetramethyl Rhodamine (TMR) (Q) (mAb SP31-TMR Conjugate)

3.5 μl of a solution of TMR (MW 527.5) (5-(and-6)-carboxytetramethylrhodamine, succinimidyl ester; sold under the reference C1171 by the company Molecular Probes, Inc., at 50 mg/ml in DMF (176 μg; 333 nmol)) are added to 880 μl of mAb SP31 at 1.136 mg/ml (1 mg corresponds to 6.67 nmol) in 0.1 M phosphate buffer, pH 7.4. After agitation at 4° C. for 16 hours, 100 μl of 1 M Tris-HCl buffer, pH 9, are added and the agitation is continued for 16 hours at 4° C., in order to inactivate the residual active ester functions. Finally, the labeled antibody is separated from the free TMR by exclusion chromatography on G-25 gel (Sephadex G-25 fine, Amersham Biosciences) in 0.1 M potassium phosphate buffer, pH 7.4, containing 0.01% of sodium azide. The fractions corresponding to the antibody are combined and the absorbance of the solution at 280 and at 555 nm is measured using a cuvette with an optical path length of 1 cm.

The antibody concentration is calculated according to the following equation:
Concentration (M)=[A₂₈₀·(A₅₅₅×0.3)]/ε

in which:

A₂₈₀ is the absorbance at 280 nm

A₅₅₅ is the absorbance at 555 nm

0.3 is the ratio of absorbance of the TMR at 280 nm and at 555 nm

ε is the molar extinction coefficient (for IgG, ε=203 000 cm⁻¹ M⁻¹).

A labeled antibody concentration of 1.36 μM is obtained.

The degree of labeling can be calculated according to the following equation:
number of molecules of TMR per molecule of antibody=A₅₅₅/(65 000×antibody concentration)

in which the number 65 000 corresponds to the molar extinction coefficient at 555 nm of the TMR.

3.6 mol of TMR per mole of antibody are obtained.

2) Preparation of the Solid Support: Functionalization of the Surface with Neutravidin

In order to be able to immobilize the tripod Y prepared above in example 4 on a solid support, the latter is, in a first step, functionalized with neutravidin.

a) Reagents Used

Saturation Buffer:
0.1 M potassium phosphate buffer, pH 7.4;
0.15 M NaCl;
bovine serum albumin (BSA) sold under the reference A-7906 by the company Sigma: 0.1%;
0.01% sodium azide.

Washing Buffer:
0.01 M potassium phosphate buffer, pH 7.4;
Tween® 20: 0.05%.

100 μl of a solution of neutravidin (sold under the reference 31 000 by the company Pierce) at 5 μg/ml in a 0.1 M potassium phosphate buffer, pH 7.4 are deposited into the wells of black microplates sold under the name High Bind Matrix by the company VWR (reference 80120696). After 16 hours at ambient temperature, the plates are washed with washing buffer and then saturated by depositing 300 μl/well of saturation buffer for 16 hours at ambient temperature. The plates are stored with the same buffer (300 μl/well) at 4° C.

3) Attachment of the Tripod Y in Accordance with the Invention (Labeled with Fluorescein) to the Microtitration Plates

A dilution range for the tripod Y (labeled with fluorescein) synthesized above in example 4, in 0.1 M potassium phosphate buffer, pH 7.4, is prepared by producing a first point of the range at a concentration of 0.5 μM, and then 4 successive ⅓ dilutions (i.e. 0.167 μM; 0.055 μM; 0.0185 μM and 6.2×10⁻³ μM). 100 μl of each concentration in duplicate are deposited into wells coated with neutravidin or for which only the saturation step is performed (negative control). After incubation for 3 hours at ambient temperature with agitation, the plates are washed with the washing buffer (5 times) and then 100 μl of 0.1 M potassium phosphate buffer, pH 7.4, are deposited. The plate is read with an analyst AD system fluorimeter (LJL Biosystem) for reading microplates, at an excitation wavelength of 485 nm and at an emission wavelength of 530 nm, which are the excitation and emission wavelengths corresponding to fluorescein.

Results

The curves obtained with neutravidin (black square) and without neutravidin (black triangle: control) are represented in the attached FIG. 2, in which the fluorescence (in arbitrary units) is expressed as a function of the concentration of tripod Y in accordance with the invention (μM).

These results show an increase in fluorescence that is proportional to that of the concentration of tripod in accordance with the invention, up to a concentration of 0.17 μM, and then a plateau corresponding to the saturation of the sites for binding of the neutravidin by the tripod. In the absence of immobilized neutravidin, no increase in fluorescence is observed.

In the examples that follow, the tripod Y of the example will be used at a concentration of 20 nm, which makes it possible, with regard to these results, to obtain a sufficient signal.

4) Saturation of the Solid Phase with the mAb SP31-TMR so as to Form a Complex C

A dilution range of the mAb SP31-TMR in 0.1 M potassium phosphate buffer, pH 7.4, is prepared by producing a first point of the range at a concentration of 100 nM and then 6 three-fold dilutions. 100 μl of each concentration in duplicate are deposited with 100 μl of a solution of the tripod Y of example 4 (solution at 20 nM in 0.1 M potassium phosphate buffer, pH 7.4) are deposited into the wells of a microplate. After incubation at 4° C. for 16 hours, the microplates are washed with washing buffer (5 times), and then 100 μl of 0.1 M potassium phosphate buffer, pH 7.4, are deposited and the fluorescence is measured.

The results obtained are represented in the attached FIG. 3, in which the fluorescence measured (in arbitrary units) is expressed as a function of the concentration of mAb SP31-TMR (in nM).

These results show that, from an antibody concentration of 1 nM, the fluorescence decreases proportionally to the concentration of mAb SP31-TMR, up to a maximum inhibition of 70% obtained using a concentration of mAb SP31-TMR of 33nM. These results demonstrate that:

i) the antibody complexes with the tripod Y in accordance with the invention by recognizing the epitope,

ii) the configuration of the tripod Y in accordance with the invention allows resonance energy transfer from the fluorescein to the TMR, which results in a decrease in the fluorescence of the fluorescein.

5) Detection of Substance P on a Solid Support

A dilution range of substance P is prepared in 0.1 M potassium phosphate buffer, pH 7.4, by producing a first point of the range at a concentration of 200 nM and then 6 three-fold dilutions. 100 μl of the tripod prepared in example 4 (20 nM) and 100 μl of mAb SP31-TMR (30 nM) (or 100 μl of 0.1 M potassium phosphate buffer, pH 7.4, for the negative control wells) are then deposited into each well. After incubation at ambient temperature for 5 hours with agitation, the plates are washed with washing buffer (5 times) and then 100 μl of 0.1 M potassium phosphate buffer, pH 7.4, are deposited and the fluorescence measured.

The results obtained are represented in the attached FIG. 4, in which the percentage inhibition by the substance P of the decrease in fluorescence caused by the mAb SP31-TMR (% ID) is expressed as a function of the concentration of substance P (in nM).

This percentage inhibition is calculated using the following equation:
% ID=[(Flₓ−Fl₀)/(Fl−Fl₀)]×100

in which:

Flₓ corresponds to the fluorescence measured in the presence of substance P at the concentration x;

Fl corresponds to the fluorescence measured in the absence of mAb SP31-TMR and of substance P;

Fl₀ corresponds to the fluorescence measured in the presence of mAb SP31-TMR and absence of substance P.

These results show, as expected, that the increase in concentration of substance P induces an increase in fluorescence (corresponding to the inhibition of the decrease) of the fluorescein. A standard curve is thus obtained, for which the limit of detection of substance P is approximately 2 nM.

6) Solid-Phase Regeneration Test

Three dilutions of substance P (respectively 1, 0.1 and 0.01 μM) are prepared in 0.1 M potassium phosphate buffer, pH 7.4. 100 μl of the tripod Y as prepared above in example 4 (20 nM) and 100 μl of mAb SP31-TMR (50 nM) (or 100 μl of 0.1 M potassium phosphate buffer, pH 7.4, for the negative control wells: Fl₀ wells) are then deposited into each well. The mAb SP31-TMR is used at 50 nM in order to complex all the tripods present on the solid phase. After incubation at 4° C. for 16 hours, the plates are washed with washing buffer (5 times) and then 100 μl of each concentration of substance P in triplicate are deposited (Flₓ wells). The Fl and Fl₀ wells are also prepared in triplicate. After incubation at ambient temperature for 5 hours with agitation, the plates are washed with washing buffer (5 times) and then 100 μl of 0.1 M potassium phosphate buffer, pH 7.4, are deposited and the fluorescence is measured (first assay).

The plates are again washed in the same manner as above, and 100 μl of mAb SP31-TMR (50 nM) (or 100 μl of 0.1 M potassium phosphate buffer, pH 7.4, for the Fl wells) are incubated for 16 hours at 4° C. The plates are again washed (5 times), and then 100 μl of 0.1 M potassium phosphate buffer, pH 7.4, are deposited and the fluorescence is measured (plate regeneration step).

The plates are again washed (5 times) and either 100 μl of potassium phosphate buffer, for the Fl and Fl₀ wells, or 100 μl of substance P (at 1, 0.1 or 0.01 μM) are deposited. After incubation at ambient temperature for 5 hours with agitation, the plates are washed (5 times), and then 100 μl of 0.1 M potassium phosphate buffer, pH 7.4, are deposited and the fluorescence is measured (second assay).

The results obtained are represented in the attached FIG. 5, in which the fluorescence (in arbitrary units) measured for the Fl, Fl₀ and Flₓ wells is expressed for the first and second assays and also for the measurement carried out after the regeneration step.

These results demonstrate that:

i) the various steps of the method of detection (deposits, washes) do not bring about dissociation of the tripod Y in accordance with the invention from the solid phase: the fluorescence measured in the Fl wells is constant;

ii) the depositing of the mAb SP31 allows an even regeneration of the solid phase: same fluorescence values for all the Flₓ and Fl₀ wells;

iii) complete regeneration is obtained: the fluorescence measured for the Flₓ wells after the regeneration step is similar to that of the Fl₀ wells carried out in the first assay;

iv) this regeneration step is compatible with a second assay carried out subsequently on the same solid phase, since the same fluorescence values are obtained for identical substance P concentrations.

7) Demonstration of the Feasibility of Repeated Assays on the Same Solid Phase

Three dilutions of substance P (1, 0.1 and 0.01 μM) are prepared in 0.1 M potassium phosphate buffer, pH 7.4.

i) 100 μl of the tripod prepared above in example 4 (20 nM) and 100 μl of mAb SP31-TMR (50 nM) or 100 μl of 0.1 M potassium phosphate buffer, pH 7.4 (negative control: Fl₀ wells) are deposited into each well of a microplate. The mAb SP31-TMR is used at the concentration of 50 nM in order to complex all the tripods present on the solid phase. After incubation at 4° C. for 16 hours, the microplate is washed with washing buffer (5 times) and then 100 μl of each concentration of substance P are deposited, in triplicate. The Fl and Fl₀ wells are also prepared in triplicate. After incubation at ambient temperature for 5 hours with agitation, the microplate is washed (5 times) and then 100 μl of 0.1 M potassium phosphate buffer, pH 7.4, are deposited and the fluorescence is measured (first assay).

ii) The plate is again washed (5 times) and 100 μl of mAb SP31-TMR (50 nM) (or 100 μl of 0.1 M potassium phosphate buffer, pH 7.4, for the Fl wells) are incubated at 4° C. for 16 hours (regeneration step).

iii) The plate is again washed (5 times) and either 100 μl of phosphate buffer, for Fl and Fl₀, or 100 μl of substance P (at 1, 0.1 and 0.01 μM) are deposited. After incubation at ambient temperature for 5 hours with agitation, the plate is washed (5 times), and then 100 μl of phosphate buffer are deposited and the fluorescence is measured (second assay).

For the subsequent assays steps ii) and iii) are repeated, varying the wells in which the phosphate buffer, for Fl₀, and the various concentrations of substance P are deposited.

11 successive assays are thus carried out on the same solid phase.

The results obtained are represented in the attached FIGS. 6 and 7.

In FIG. 6, the fluorescence (in arbitrary units) corresponds, for each Fl, Fl₀ and Flₓ well, to the mean of the 11 assays carried out. In FIG. 7, the fluorescence (in arbitrary units) corresponds to the values obtained for each of the 11 assays carried out for the Fl, Fl₀ and Flₓ wells with x=1 μM of substance P.

These results show that, for each concentration of substance P, the fluorescence values are regular and greater than those obtained in the absence of substance P. In addition, the fluctuations observed are random: there is no gradual decrease or increase in the fluorescence over the course of the assays.

These results also show that the method of detection in accordance with the present invention allows repeated assaying of an analyte on a solid phase, it being possible for each of the wells (or reaction site) to be regenerated in the equivalent manner and to allow a subsequent further detection.

### EXAMPLE 6

### Detection of Substance P and Regeneration in a Capillary

1) Procedure

a) Preparation of the Reagents Used

Labeling of the tripod with a fluorescent molecule:

The labeling of the substance P analog of sequence SEQ ID No. 2 (as described above in example 4) with a fluorescent molecule sold under the brand name Alexa Fluor® 532 Protein Labeling Kit, reference A-10236 (Molecular Probes) is carried out according to the protocol described above in example 4. A tripod-Alexa 532 is obtained.

Preparation of a receptor-Q: labeling of the mAb SP31 antibody with a fluorescent molecule (mAb SP31-Alexa 647 conjugate):

The labeling of the mAb SP31 with a fluorescent molecule sold under the brand name Alexa® Fluor 647 Succinimidyl Esters, reference A-20006 (Molecular Probes), is carried out according to the protocol described above in example 5, step 1). An mAb SP31-Alexa 647 labeled antibody is obtained.

Preparation of capillaries:

Capillaries made of borosilicate with a square cross section are used (inside edge=0.5 mm, outside edge=1 mm, length=25 mm) (Wale Apparatus). The capillaries are functionalized with neutravidin and then incubated in a solution of tripod-Alexa 532 (solutions at 5 μM) overnight at 4° C.

b) Experimental Protocol

The functionalized capillary is connected, via Manifold tubing with an inside diameter of 0.51 mm (Bioblock), to a Gilson pump, allowing the successive passage of various solutions.

A fluorescence microscope (excitation by means of a mercury vapor lamp) equipped with an HisIs 23 cooled CCD camera (Europixel, Italy) and a shutter that are computer-controlled is used to measure the fluorescence.

The image acquisition is carried out on the same area of the capillary throughout the experiment, with an exposure time of 50 msec, using a 530-560 nm excitation filter and a 575-645 nm emission filter.

After washing of the capillary with washing buffer as described above in example 5, the functionalized capillary is filled with 0.1 M phosphate buffer, pH 8.5, and the initial fluorescence at t=0 is read.

A solution of mAb SP31-Alexa 647 at 2.62 μM is then introduced into the capillary. After 10 minutes, the capillary is washed (washing buffer for 2 minutes at a flow rate of 35 μl/min) and then filled with 0.1 M phosphate buffer, pH 8.5, and the fluorescence after quenching is measured.

A solution of substance P at 10 nmol/ml is subsequently introduced for 80 minutes. After a first measurement at t=15 seconds, the measurements are carried out every 5 minutes.

After washing of the capillary (washing buffer and then 0.1 M phosphate buffer, pH 8.5, for 2 minutes for each, at 35 μl/min), the solid phase is regenerated by means of a further incubation with mAb SP31-Alexa 647. The experiment is repeated 3 times in this way.

2) Results

The results obtained in this experiment are represented in the attached FIG. 8, in which the fluorescence (in arbitrary units) is expressed as a function of time (minutes).

These results show that the extinction (quenching) of the fluorescence of the tripod-Alexa 532 by the mAb SP31-Alexa 647 (zone 1 of the curve), the increase in fluorescence in the presence of substance P (zone 2 of the curve) and the step consisting of regeneration by introducing mAb SP31-Alexa 647 (zone 3 of the curve) remain effective on solid supports such as capillaries and with luminescent groups (L) and quenching groups (Q) other than those used in example 5.

1-27. (canceled)

28. A method for detection of an analyte a in a fluid sample, comprising the following steps:
1) saturating a solid support comprising, on at least part of its surface, at least one trifunctional reagent (tripod Y) comprising the following three functional poles:
i) a luminescent group (L),
ii) a molecule (B) selected from the group consisting of the analyte a, an analog of the analyte a or a fragment of the analyte a; and
iii) a function that provides attachment of the trifunctional reagent to the surface of the solid support,
with a receptor for the analyte a, the receptor being labeled with a compound (Q) (receptor-Q) that quenches the luminescence of the group L, so as to form a complex C between the molecule (B) and the receptor-Q;
2) bringing the solid support obtained in step 1) into contact with a fluid sample that may comprise the analyte a to be detected;
3) measuring the intensity of the signal emitted by the group L, which is proportional to the amount of analyte a present in the fluid sample; and
4) regenerating the solid support by bringing the solid support into contact with the receptor-Q.

29. The method as claimed in claim 28, wherein several types of tripods Y that differ from one another through the nature of the molecule (B) that they comprise are attached to distinct and known zones of the solid support.

30. The method as claimed in claim 28, wherein step 3) and step 4) are carried out continuously.

31. The method as claimed in claim 28, wherein the solid support is selected from the group consisting of glasses, plastics, ceramics, metals and metalloids.

32. The method as claimed in claim 28, wherein the solid support is in the form of a tube, a capillary, a plate or a bead.

33. The method as claimed in claim 28, wherein the fluid sample consists of water, a liquid biological medium, or a liquid medium comprising dissolved gaseous molecules or molecules originating from solid samples.

34. The method as claimed in claim 28, wherein the intensity of the signal emitted during step 3) is determined by a luminescence detector.

35. The method as claimed in claim 28, wherein the complex C formed at the end of the saturation in 1) is selected from the group consisting of complexes of formula (I) below: 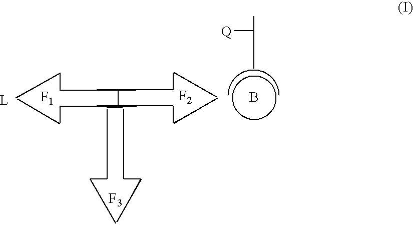
wherein:
the arrows represent the structure of the backbone of the tripod Y, which is a linker arm consisting of a peptide, nucleotide or glucoside chain or of a saturated or unsaturated, linear or branched hydrocarbon-based chain; the chains being optionally substituted, interrupted and/or ended with one or more hetero atoms, such as N, O or S, and/or with one or more amino acids, and comprising three reactive chemical functions F₁, F₂ and F₃;
L represents a luminescent group covalently bonded to the tripod Y by the reactive chemical function Fl;
B represents an analyte a, a structural analog of an analyte a or a fragment of an analyte a to which is noncovalently and reversibly attached a receptor specific for the analyte a, the receptor being labeled with a compound Q; the molecule (B) being covalently bonded to the tripod Y by the reactive chemical function F₂;
Q represents a compound that quenches the luminescence of the group L; and
F₃ represents a reactive chemical function that can allow the attachment of the tripod Y to the surface of the solid support.

36. The method as claimed in claim 35, wherein the functions F₁, F₂ and F₃, independently of one another, provide:
i) either a direct linkage via a corresponding chemical function present on the luminescent compound, the molecule (B) or the solid phase;
ii) or an indirect linkage, and in this case, the linkage is carried out by coupling, to at least one of the functions F₁, F₂ and/or F₃, a molecule M₁ forming a complex with a molecule M₂ attached beforehand to at least part of the surface of the solid phase, to the molecule (B) and/or to the luminescent group.

37. The method as claimed in claim 35, wherein the functions F₁, F₂ and F₃, which may be identical or different, are selected from the group consisting of: thiols; amines; alcohols; acid functions; esters; isothiocyanates; isocyanates; acylazides; sulfonyl chlorides; aldehydes; glyoxals; epoxides; oxiranes; carbonates; imidoesters; carbodiimides; maleimides; nitriles; aziridines; acryloyl; halogenated derivatives; disulfide groups; phosphorus-containing groups; diazo; carbonyldiimidazole; hydrazides; arylazides; hydrazines; diazirines; magnesium compounds; lithium compounds; cuprates; zinc compounds and unsaturated systems.

38. The method as claimed in claim 37, wherein the functions F₁, F₂ and F₃ are selected from the group consisting of amine functions of formulae R—NH₂, R—NH—, (R)₃—N, R—NH—OR and NH₂—OR; alcohol functions R—OH; and halogenated groups of formula R—X with X representing a halogen atom; it being understood that, in the formulae, R represents an alkyl, aryl, vinyl or allyl radical.

39. The method as claimed in claim 28, wherein the luminescent group is selected from the group consisting of fluorescein and its derivatives; rhodamine and its derivatives; diaminidophenyl indo; acridine; fluorescent dyes with reactive amines; eosin; and erythrosine.

40. The method as claimed in claim 28, wherein the receptor is selected from the group consisting of antibodies in whole, fragmented or recombinant form, biological receptors, nucleic acids, peptide nucleic acids, lectins, transporter proteins, chelates and synthetic receptors.

41. The method as claimed in claim 28, wherein the receptor exhibits greater affinity for the analyte a than for the molecule (B).

42. The method as claimed in claim 28, wherein the quenching compound (Q) is selected from the group consisting of rhodamine and its derivatives, the fluorescent compounds mentioned in claim 12, and nonfluorescent molecules.

43. The method as claimed claim 35, wherein the complexes of formula (I) are selected wherein:
i) (B) is selected from the group consisting of peptides, proteins, oligonucleotides, sugars and peptide nucleic acids,
ii) L is fluorescein, and
iii) the backbone of the tripod Y is selected from the group consisting of the structures Y₁ to Y₃ below: 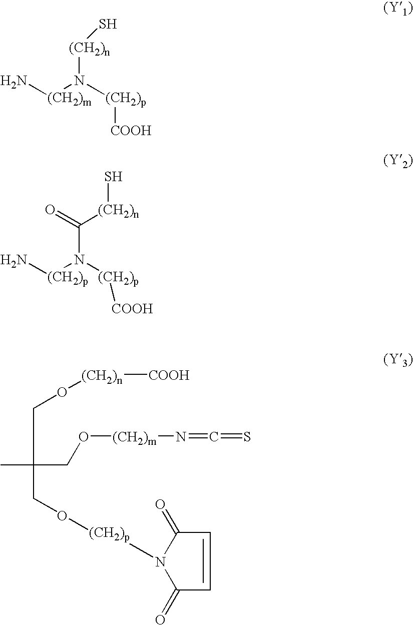
wherein n, m and p, which may be identical or different, are integers between 1 and 20 inclusive.

44. The method as claimed in claim 43, wherein structures Y₁ to Y₃ are selected from the group consisting of compounds of formulae (Y′₁) to (Y′₃) below:

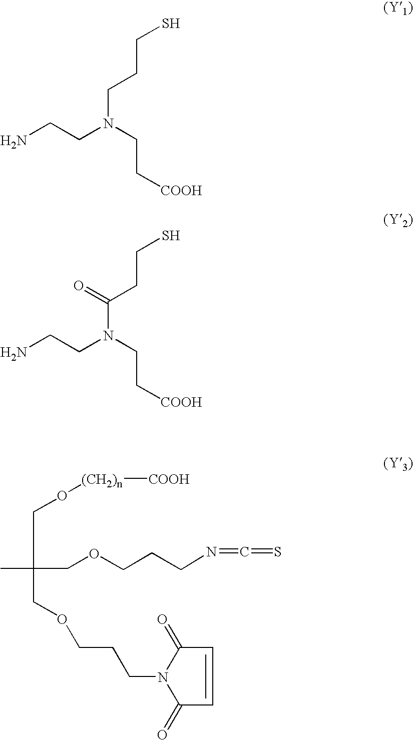

45. A complex C, wherein it corresponds to formula (I) below: 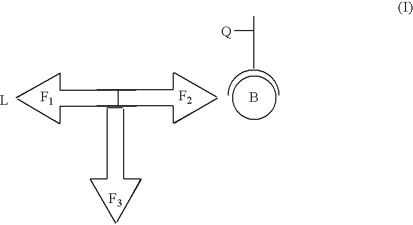
wherein L, B, Q, the arrows and F₁, F₂ and F₃ are as defined in claim 35.

46. A method for continuous heterogeneous-phase detection of an analyte a in a fluid sample, comprising detecting the analyte a in a fluid sample with at least one complex C of formula (I).

47. A device for continuous heterogeneous-phase detection of at least one analyte a in a fluid sample, wherein a fluid sample to be analyzed is integrated into a medium forming a stream that flows over at least one solid support at the surface of which is attached at least one tripod Y as defined in claim 1 and specific for the analyte a to be detected, a luminescence detector placed opposite the solid support is coupled to a valve control that is controlled by a threshold of intensity of signal emitted by the detector and which triggers, for a given period of time, the opening of a reservoir comprising a receptor-Q capable of forming a complex with the tripod Y, this reservoir being linked to the support via a feedback loop which comes in upstream of the solid support to which the tripod Y is attached, in order to saturate and/or regenerate the latter with receptor-Q by passage in the stream and complexation on the tripod Y.

48. The device as claimed in claim 47, wherein luminescence intensity values are monitored and secondarily translated into an amount of analyte a by a calculation system coupled to the luminescence detector.

49. The device as claimed in claim 47, wherein an event marker is placed in the feedback loop in order to signal a variation in intensity of the signal above a predetermined value.

50. The device as claimed in claim 47, wherein the solid support is a capillary coupled to the environment containing the sample to be analyzed, the coupling being carried out either by a round-bottomed capture flask wherein the sample sparges in a medium corresponding to that of the flow stream, or by a flexible pipe.

51. The device as claimed in claim 47, wherein the stream is entrained by the low pressure produced by a pump, a piston, or equivalent.

52. The device as claimed in claim 47, wherein the device is equipped with a round-bottomed capture flask and with a sparging system for collecting samples in gaseous form and for solubilizing the constituents to be detected that they contain.

53. A method for detecting the presence of an analyte a in a natural or industrial medium, comprising detecting the presence of an analyte a in a natural or industrial medium with the device defined in claim 47.

54. The method as claimed in claim 53, wherein the detecting is performed in lakes, rivers, swimming pools, factories, purification plants, or ventilation or air-conditioning systems.


SEQUENCE LISTING (2 sequences)

SEQ ID NO: 1 - protein, 11 aa, Artificial Sequence
  Synthetic Peptide
RPKPQQFFGLM

SEQ ID NO: 2 - DNA, 15 bp, Artificial Sequence
  Synthetic DNA
gatctgctggcatct